(19) United States
(12) Patent Application Publication
(10) Pub. No.: US 2018/0138717 A1
(43) Pub. Date: May 17, 2018

(54) POWER AND INFORMATION TRANSFER BETWEEN UNITS HAVING RELATIVE MOVEMENT

(71) Applicant: ANALOGIC CORPORATION, Peabody, MA (US)

(72) Inventors: Stephen J. QUIGLEY, East Hampstead, NH (US); Matthew HANLON, Kingston, NH (US); Louis R. POULO, Andover, MA (US)

(21) Appl. No.: 15/570,405

(22) PCT Filed: Apr. 30, 2015
(86) PCT No.: PCT/US2015/028552

§ 371 Date: Oct. 30, 2017

(51) Int. Cl. H02J 5/00; A61B 6/03; A61B 6/00; H02J 50/10; H04B 5/00; H05G 1/12; G01N 23/046

(52) U.S. Cl. CPC H02J 5/005; A61B 6/032; A61B 6/56; G01N 23/046; H04B 5/0037; H05G 1/12; H02J 50/10

(57) ABSTRACT

One or more techniques and/or systems are provided for transferring power and/or information between a stator and a rotor. The rotor includes a high voltage rectifier filter and an auxiliary regulator coupled to a power transfer apparatus (e.g., rotary transformer). The high voltage rectifier filter is configured to receive a first electrical signal, associated with a first voltage, from the power transfer apparatus and to generate a second electrical signal, associated with a second voltage, based on a frequency and/or an amplitude of the first electrical signal. The auxiliary regulator is configured to generate a third electrical signal based on the first electrical signal, where a third voltage associated with the third electrical signal is generated independent of the frequency and/or amplitude of the first electrical signal. A single bi-directional data link is used for control and/or communication between the stator and the rotor.

### BACKGROUND

The present application relates to the delivery of power and/or information between two members configured for relative movement. It finds particular application in computed tomography (CT) imaging modalities where a rotor comprising a radiation source, detector array, etc. is rotated relative to a stator, although it may find utility in other applications as well.

Systems that comprise electronic components within a moving unit often require power and/or information (e.g., data) to be provided to the moving unit. In CT imaging modalities, for example, slip-ring assemblies have traditionally been used to transfer the power and/or the information between a stator and a rotor. Slip-ring assemblies are configured to transfer power and/or information between a stator and a movable member (e.g., the rotor) and/or between two movable members through the physical contact of two materials (e.g., via a sliding contact). For example, a slip-ring attached to the stator may comprise metal brushes that are configured to physically contact an electrically conductive surface of a slip-ring attached to the movable member, allowing power and/or information to be transferred between the stator and the movable member.

In CT imaging modalities, the power and the information have conventionally been transferred via a plurality of slip rings. For example, a first slip ring supplies high voltage power (e.g., 60 kW and 100 kW of power) to a radiation source while a second slip ring supplies lower voltage power (e.g., 5 kW of power or less) to other electronic components that operate concurrently with the radiation source but may use much less power (e.g., 5 kW or less). A third slip ring may supply information between the stator and the rotor.

While the use of slip-ring assemblies has proven effective for transferring power and/or information between a stator and a movable unit (e.g., a rotor) and/or between two movable units, conventional slip-ring assemblies may generate dust or particles (e.g., as metal brushes wear), may be unreliable (e.g., again as contact surfaces, such as metal brushes, wear), and/or may be noisy (e.g., as surfaces rub against one another), which may cause interference with some procedures. Other drawbacks of slip-ring assemblies may include cost and complexity of manufacture due to special materials and/or mechanical precision that may be required. Further, these slip rings (e.g., and associated power electronics that feed the electrical signals across the slip rings) add weight (e.g., 50 pounds or more) to the rotor and/or consume valuable space on the rotor.

### SUMMARY

Aspects of the present application address the above matters, and others. According to one aspect, a computed tomography (CT) imaging modality comprises a stator, a rotor configured to rotate relative to the stator, and a rotary transformer comprising a primary winding disposed on the stator and a secondary winding disposed on the rotor. The rotary transformer is configured to deliver power between the stator and the rotor. The CT imaging modality also comprises a high voltage rectifier filter coupled to the secondary winding and configured to receive a first electrical signal from the secondary winding. The first electrical signal is associated with a first voltage and the high voltage rectifier filter is configured to generate a second electrical signal associated with a second voltage, greater than the first voltage, based on a frequency and an amplitude of the first electrical signal. The CT imaging modality comprises an auxiliary regulator coupled to the secondary winding and configured to generate a third electrical signal based on the first electrical signal. The third electrical signal is associated with a third voltage. The auxiliary regulator is configured to self-regulate the third voltage.

According to another aspect, a computed tomography (CT) imaging modality comprises a stator, a rotor configured to rotate relative to the stator, and a rotary transformer comprising a primary winding disposed on the stator and a secondary winding disposed on the rotor. The rotary transformer is configured to deliver power between the stator and the rotor. The CT imaging modality comprises a high voltage rectifier filter coupled to the secondary winding. The high voltage rectifier filter is configured to receive a first electrical signal, associated with a first voltage, from the secondary winding and step up the first voltage based on a frequency and an amplitude of the first electrical signal to generate a second electrical signal associated with a second voltage. The second voltage is greater than the first voltage. The CT imaging modality comprises an auxiliary regulator coupled to the secondary winding and configured to generate a third electrical signal based on the first electrical signal. The third electrical signal is associated with a third voltage, the third voltage may be greater than, less than, or equal to the first voltage.

According to another aspect, a system comprises a high voltage rectifier filter coupled to a secondary winding of a rotary transformer. The high voltage rectifier filter is configured to receive a first electrical signal, associated with a first voltage, from the secondary winding and step up the first voltage based on at least one of a frequency or an amplitude of the first electrical signal to generate a second electrical signal associated with a second voltage. The system comprises an auxiliary regulator coupled to the secondary winding and configured to generate a third electrical signal based on the first electrical signal. The third electrical signal is associated with a third voltage, the third voltage may be greater than, less than, or equal to the first voltage.

According to another aspect, a computed tomography (CT) imaging modality is provided. The CT imaging modality comprises a stator and a rotor configured to rotate relative to the stator. The CT imaging modality comprises a rotary transformer comprising a primary winding disposed on the stator and a secondary winding disposed on the rotor. The rotary transformer is configured to deliver power from the stator to both a high voltage rectifier filter on the rotor and an auxiliary regulator on the rotor. The CT imaging modality comprises a bi-directional link configured to transfer information between the rotor and the stator.

Those of ordinary skill in the art will appreciate still other aspects of the present application upon reading and understanding the appended description.

### FIGURES

The application is illustrated by way of example and not limitation in the figures of the accompanying drawings, in which like references indicate similar elements and in which:

FIG. 1 is a schematic block diagram illustrating an example environment wherein one or more of the provisions set forth herein may be implemented.

FIG. 2 illustrates a cross-sectional view of an example stator and a rotor comprising elements to facilitate the transfer of power and/or information between the stator and the rotor on a disk configuration, such as using at least one of a belt or a bearing.

FIG. 3 illustrates a cross-sectional view of an example stator and a rotor comprising elements to facilitate the transfer of power and/or information between the stator and the rotor on a radial configuration, such as using at least one of a belt or a bearing.

FIG. 4 illustrates an example system wherein one or more of the provisions set forth herein may be implemented, such as using a bearing.

FIG. 5 is a flow diagram illustrating an example method for supplying power to a high voltage load and to an auxiliary load.

FIG. 6 is an illustration of an example computer-readable medium comprising processor-executable instructions configured to embody one or more of the provisions set forth herein.

### DESCRIPTION

The claimed subject matter is now described with reference to the drawings, wherein like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are illustrated in block diagram form in order to facilitate describing the claimed subject matter.

Note that “noncontact,” “contactless,” and/or the like is generally used herein to refer to the ability to transfer information/data and/or power in inductive fashion between or among bodies configured for relative movement, and should not be understood to necessarily preclude possible contact between or among such bodies for other purposes, including, but not limited to, electrostatic discharge, mechanical drive or support, braking and/or safety mechanisms, for example.

It should also be noted that in the present disclosure, except where otherwise clear from context, “gap” and “airgap” are used more or less interchangeably. Additionally, although “airgap” may be used herein, it should be understood that such gaps are not necessarily limited to air. For example, vacuum, oil, and/or other fluid and/or gas, and/or sliding and/or roller bearings or other such contrivances are contemplated to completely or partially fill spaces to facilitate relative movement.

The present disclosure relates to a contactless power and/or information delivery system configured to transfer power and/or information between a stator and a rotor configured for movement relative to the stator. It finds particular applicability in the context of computed tomography (CT) imaging, where power is transferred from a stationary side of the CT imaging modality to a rotating side of the CT imaging modality. In some embodiments, information may be transferred bi-directionally between the stator and the rotor.

In some embodiments, the system is configured to supply power to one or more high voltage loads and one or more auxiliary loads of (e.g., disposed on) the rotor via a rotary transformer formed by or between the stator and the rotor. The rotary transformer is configured to induce merely one electrical signal on a secondary side of the rotary transformer, and typically comprises a single primary winding and a single secondary winding. In some embodiments, a number of turns in the secondary winding are matched to a number of turns in the primary winding. In some embodiments, the number of turns in the secondary winding is greater than or less than the number of turns in the primary winding.

In the context of CT imaging, the system may be configured to concurrently supply power to a high voltage load(s), such as a radiation source (e.g., which may operate at 60 kW or more of power) and to an auxiliary load(s), such as control electronics, spine heater, tube heat exchanger, anode drive, etc. (e.g., which may operate at 5 kW or less of power) via the rotary transformer. To regulate the voltage applied to the high voltage loads and the auxiliary loads, an electrical signal generated at the secondary side, or secondary winding, of the rotary transformer is transmitted to a high voltage rectifier filter, coupled to the high voltage load(s), to generate a high voltage signal. The electrical signal is also transmitted to an auxiliary rectifier filter and/or to an auxiliary regulator, coupled to the auxiliary load(s), to generate a lower voltage signal. In some embodiments, the high voltage signal is regulated by controlling an amplitude and/or a frequency of the electrical signal generated at the secondary side of the rotary transformer. In some embodiments, the auxiliary regulator self-regulates the lower voltage signal such that the lower voltage signal applied to the auxiliary load(s) is independent of the amplitude and the frequency of the electrical signal generated at the secondary side of the rotary transformer.

It will be appreciated that the terms low voltage, lower voltage, and the like are intended to be interpreted relative to the terms higher voltage, high voltage, and the like, and are not intended to be interpreted in a limiting manner such as necessarily specifying particular voltages and/or particular voltage ranges. For example, the auxiliary load may be configured to operate at voltages that have traditionally been considered low voltages (e.g., below 600 V), where the high voltage load may be configured to operate at a higher voltage than the auxiliary load. In yet another embodiment, the auxiliary load and the high voltage load may be configured to operate at what have traditionally been considered high voltages (e.g., 600 V or more), where the auxiliary load may be configured to operate at a lower voltage (e.g., but still a high voltage) than the high voltage load. In yet another embodiment, the auxiliary load may be configured to operate at a voltage that has traditionally been considered a low voltage (e.g., less than 600 V) and the high voltage load may be configured to operate at a voltage that has traditionally been considered a high voltage (e.g., equal to or greater than 600 V).

While the applicability of features described herein are described with respect to a CT imaging modality, it will be appreciated that the instant disclosure, including the scope of the claims, is not intended to be limited to such a system. That is, to the extent practical, the techniques and/or systems described herein may be used with any applications where it may be desirable to transfer power between two or more units configured for relative motion.

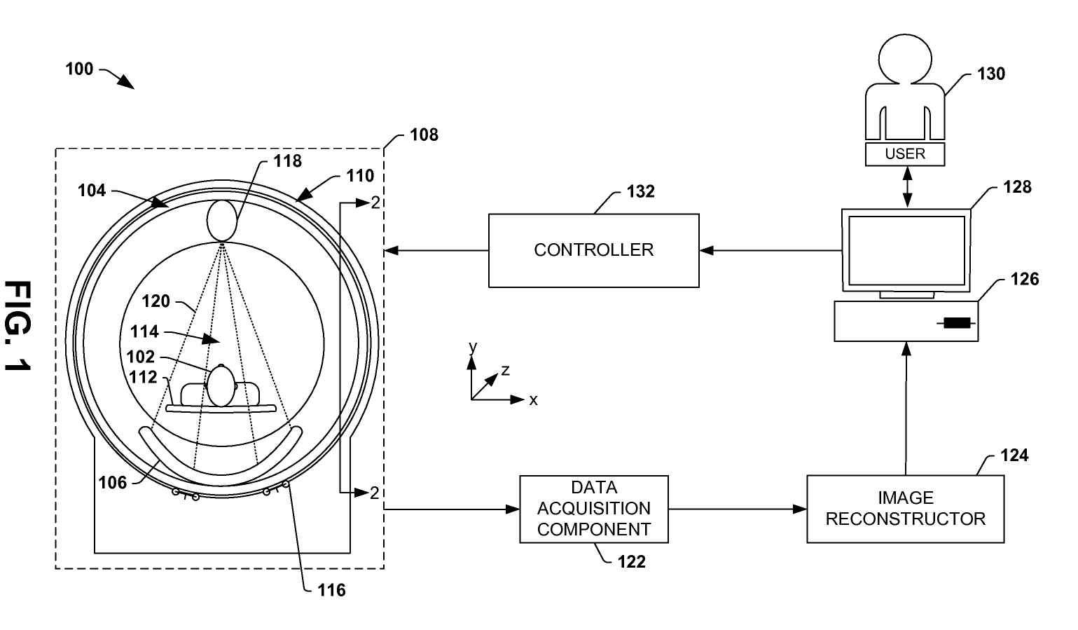

FIG. 1 is an illustration of an example environment 100 wherein a system may be configured to transfer power between a stator 110 and a rotor 104 of an examination unit 108 that is configured to examine one or more objects 102.

In particular, FIG. 1 illustrates an example CT imaging modality having a system as described here. It will be appreciated that while the example environment 100 illustrates a CT imaging modality, the instant application, including the scope of the claims, is not intended to be interpreted as applying merely within the context of CT imaging. Moreover, the example environment 100 merely illustrates an example schematic and is not intended to be interpreted in a limiting manner, such as necessarily specifying the location, inclusion, and/or relative arrangement of the components described herein. For example, a data acquisition component 122 as illustrated in FIG. 1 may be disposed on the rotor 104 of the examination unit 108, or more particularly may be part of a detector array 106, for example.

During an examination of the object(s) 102, the object(s) 102 can be placed on a support article 112, such as a bed or conveyor belt, for example, that is selectively positioned in an examination region 114 (e.g., a hollow bore in the rotor 104), and the rotor 104 can be rotated about the object(s) 102 by a rotator 116, such as a motor, drive shaft, chain, roller truck, etc. During at least portions of the examination (e.g., such as when radiation 120 is being emitted by a radiation source 118), varying amounts of power may be supplied to various components disposed on the rotor 104 via a rotary transformer or other power transfer apparatus as described in more detail below. For example, a high voltage may be applied to the radiation source 118 via the rotary transformer while a lower voltage is concurrently applied to other components of (e.g., disposed on) the rotor 104, such as to the detector array 106.

As illustrated, the rotor 104 may surround a portion of the examination region 114 and may comprise or have disposed thereon, among other things, one or more radiation sources 118 (e.g., an ionizing radiation source, such as an x-ray source or a gamma source) and the detector array 106, which is mounted on a substantially diametrically opposite side of the rotor 104 relative to the radiation source 118. It will be appreciated that other components (not shown) such as, but not limited to, control electronics, spine heater, tube heat exchanger, and/or an anode drive may also be disposed on the rotor 104. These other components are, at times, referred to as auxiliary components or an auxiliary load and typically operate at a substantially lower voltage (e.g., 400 V or less) than the radiation source(s) 118, which may operate at a voltage of 60 kV or more, and may correspond to the high voltage load.

A typical examination unit 108 generally operates under at least two operating modes. During a first operating mode, which may be referred to as a preparation mode, power is generally supplied to the auxiliary component(s) via the rotary transformer to prepare for an examination of the object. Once the auxiliary component(s) is prepared for an examination (e.g., an anode drive is rotating an anode of the radiation source(s) 118, a heat exchanger is operational, etc.), the examination unit 108 transitions to a second operating mode, which may be referred to as a shoot mode, and radiation 120 may be emitted from the radiation source(s) 118 to examine the object 102. It will be appreciated that during the shoot mode, power may be supplied to both the auxiliary component(s) (e.g., the auxiliary load) and the radiation source(s) (e.g., the high voltage load) via the rotary transformer. Thus, as will be described in more detail below, the rotary transformer or other power transfer apparatus is configured to supply the auxiliary component(s) with low voltage power and to supply the radiation source(s) 118 with higher voltage power substantially concurrently.

During an examination of the object(s) 102, the radiation source(s) 118 emits fan or cone shaped radiation configurations into the examination region 114. It will be appreciated that such radiation 120 may be emitted substantially continuously and/or may be emitted intermittently (e.g., a short pulse of radiation 120 is emitted followed by a resting period during which the radiation source 118 is not activated).

As the emitted radiation 120 traverses the object(s) 102, the radiation 120 may be attenuated differently by different aspects of the object(s) 102. Because different aspects attenuate different percentages of the radiation 120, an image(s) may be generated based upon the attenuation, or variations in the number of radiation photons that are detected by the detector array 106. For example, more dense aspects of the object(s) 102, such as a bone or metal plate, may attenuate more of the radiation 120 (e.g., causing fewer radiation photons to strike the detector array 106) than less dense aspects, such as skin or clothing.

The detector array 106 is configured to directly convert (e.g., using amorphous selenium and/or other direct conversion materials) and/or indirectly convert (e.g., using a scintillator and photodetectors and/or other indirect conversion materials) detected radiation into analog signals that can be transmitted from the detector array 106 to a data acquisition component 122 configured to convert the analog signals output by the detector array 106 into digital signals and/or to compile signals that were transmitted within a predetermined time interval, or measurement interval, using various techniques (e.g., integration, photon counting, etc.). Such a measurement interval can be referred to as a “view” and generally reflects signals generated from radiation 120 that was emitted while the radiation source(s) 118 was at a particular angular range relative to the object(s) 102. Based upon the compiled signals, the data acquisition component 122 can generate projection data indicative of the compiled signals, for example.

The example environment 100 further comprises an image reconstructor 124 configured to receive the projection space data that is output by the data acquisition component 122 and to generate image space data from the projection data using a suitable analytical, iterative, and/or other reconstruction technique (e.g., backprojection reconstruction, tomosynthesis reconstruction, iterative reconstruction, etc.). In this way, the data is converted from projection space to image space, a domain that may be more understandable by a user 130 viewing the image(s), for example.

The example environment 100 also comprises a terminal 126, or workstation (e.g., a computer), configured to receive the image(s), which can be displayed on a monitor 128 to the user 130 (e.g., security personnel, medical personnel, etc.). In this way, a user 130 can inspect the image(s) to identify areas of interest within the object(s) 102. The terminal 126 can also be configured to receive user input which can direct operations of the object examination unit 108 (e.g., a speed of a conveyor belt, activation of the radiation source(s) 118, etc.).

In the example environment 100, a controller 132 is operably coupled to the terminal 126. In one example, the controller 132 is configured to receive input from the terminal 126, such as user input for example, and to generate instructions for the object examination unit 108 indicative of operations to be performed. For example, the user 130 can desire to reexamine the object(s) 102, and the controller 132 can issue a command instructing the support article 112 to reverse direction (e.g., bringing the object(s) 102 back into an examination region 114 of the object examination unit 108).

As will be described in more detail below, power, commands, status information, and/or other information may be transmitted between the rotor 104 and the stator 110 via electromagnetic couplings formed between the rotor 104 and the stator 110.

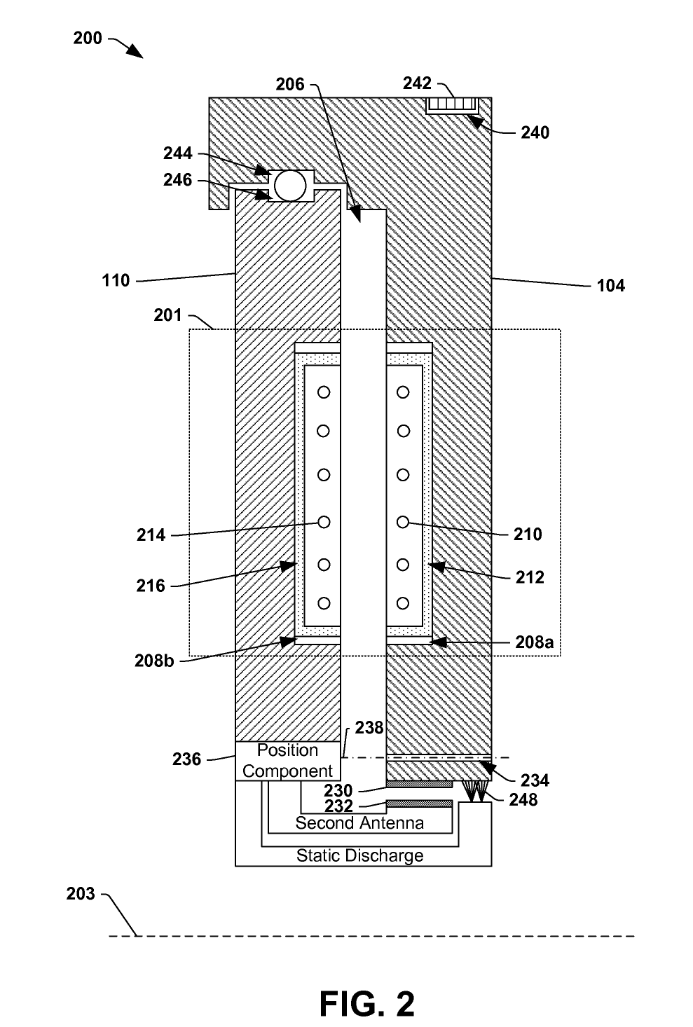

FIG. 2 illustrates a cross-sectional view 200 of an example rotor 104 and a stator 110 separated by a substantially planar airgap 206, wherein the rotor 104 is configured to rotate about an axis of rotation 203. The rotor 104 comprises a channel 208a disposed on an axial surface of the rotor 104 and the stator 110 comprises a channel 208b disposed on an axial surface of the stator 110. Electrically conductive elements (e.g., solid or braided wires) may be disposed within the channels 208a, 208b to provide for contactless transfer of power across the airgap 206. In this way, power may be inductively transferred across the airgap 206, as opposed to using slip-rings to transfer such power, for example.

A rotary transformer 201 at least partially defined by the electrically conductive elements is configured to facilitate the transfer of high voltage power and lower voltage power (e.g., auxiliary power) between the rotor 104 and the stator 110. The stator 110 may comprise an electrically conductive (annular shaped) primary winding 214 and a core 216 (e.g., a ferromagnetic core comprising manganese-zinc, nickel-zinc, etc.). The primary winding 214 is disposed within a first channel 208b of the stator 110, and the core 216 is disposed between the primary winding 214 and a surface of the first channel 208b.

The rotor 104 may comprise an electrically conductive (annular shaped) secondary winding 210 and a core 212 (e.g., a ferromagnetic core comprising manganese-zinc, nickel-zinc, etc.). The secondary winding 210 is disposed within a first channel 208a of the rotor 104, and the core 212 is disposed between the secondary winding 210 and a surface of the first channel 208a. The secondary winding 210 of the rotor 104 may be configured to inductively generate power via electro-magnetic fields produced by current flowing through the primary winding 214 of the stator 110. Thus, the rotary transformer 201 facilitates creation of an inductive coupling between the stator 110 and the rotor 104 through which power is transferred from the stator 110 to the rotor 104.

It may be appreciated that in the illustrated embodiment, the primary winding 214 of the stator 110 and the secondary winding 210 of the rotor 104 respectively comprise six turns (e.g., the wire is looped in respective channels 208a, 208b six times). However, in other embodiments, the primary winding 214 and/or secondary winding 210 may comprise more turns or a fewer number of turns than the illustrated number of turns. Moreover, the number of turns in the primary winding 214 of the stator 110 may be different than the number of turns in the secondary winding 210 of the rotor 104 (e.g., so that voltage step-ups or voltage step-downs occur between the primary winding 214 and the secondary winding 210).

In some embodiments, to facilitate the transfer of information (e.g., data transfer, image data, communication data, etc.) between the rotor 104 and the stator 110, a bi-directional link may be formed by the rotor 104 and the stator 110. For example, the rotor 104 may comprise a first antenna 230 of the bi-directional link and the stator 110 may comprise a second antenna 232 of the bi-directional link. Typically, the first antenna 230 and/or the second antenna 232 are configured for transfer speeds that range between 500 megahertz and several gigahertz, although transfer speeds outside of this range are also contemplated. For example, U.S. patent application Ser. No. 13/453,203, entitled “Contactless Communication Signal Transfer” and assigned to Analogic Corporation, describes a wide-frequency bandwidth antenna assembly configured to transfer image data produced by a detector array to a receiver on the stationary side of an imaging modality and is incorporated herein by reference. As another example, U.S. Pat. No. 5,557,026, entitled “Apparatus for transferring data to and from a moving device” and assigned to Analogic Corporation, describes an antenna assembly for transferring image data between a moving device and a stationary device and is incorporated herein by reference. Typically, at least one of the first antenna 230 or the second antenna 232 extends along a circumference of the rotor 104 and/or the stator 110 while the other antenna may extend along merely a portion of the circumference.

As illustrated, the first antenna 230 is positioned on a different surface of the rotor 104 than the windings/elements described above for facilitating the transfer of power. For example, in the illustrated arrangement 200, the elements are positioned on the axial surface and the first antenna 230 is positioned on the radial surface. However, in other embodiments, at least some of the windings/elements may be positioned on a same surface as the first antenna 230. Moreover, while FIG. 2 illustrates the first antenna 230 as protruding from a surface of the rotor 104, in another embodiment, the rotor 104 may comprise a channel and the first antenna 230 may be positioned within the channel (e.g., to improve alignment of the first antenna 230 relative to the rotor 104). These features may also find applicability with respect to the second antenna 232 and the stator 110.

In some embodiments, information transfer between the first antenna 230 and the second antenna 232 may be bi-directional. In other embodiments, the first antenna 230 and the second antenna 232 may support uni-directional communication. Accordingly, in some embodiments, the bi-directional link may comprise another set of antennas (e.g., formed within a same antenna structure as the first set of antennas) disposed on the rotor 104 and the stator 110 to facilitate bi-directional communication between the stator 110 and the rotor 104 (e.g., where one set of antennas facilitates communication in a first direction and another set of antennas facilitates communication in a second direction opposite the first direction).

To facilitate ascertaining a rotation angle of the rotor 104 (e.g., relative to a zero-degree reference) during the rotation of the rotor 104, the rotor 104 may further comprise one or more positioning elements 234 (e.g., slits, cavities, holes, notches, channels, etc.) and the stator 110 may comprise a position component 236 configured to determine the rotation angle of the rotor 104 based upon the one or more positioning elements (or vice-versa). For example, in one embodiment, one or more bores 234 may be formed within the rotor 104 and the positioning component 236 may be configured to emit a light beam, infrared beam, or other electromagnetic radiation 238 in the direction of the bores 234 to determine the rotation angle of the rotor 104. Typically, the bores 234 are spaced evenly around the rotor 104, and when the electromagnetic radiation 238 encounters a bore 234 (e.g., or when electromagnetic radiation is not reflected back to the position component 236), it may be determined that the rotor 104 has rotated a predetermined distance. For example, in one embodiment, the rotor 104 may comprise 360 bores spaced one-degree apart. As such, the position component 236 may determine that the rotor 104 has rotated one-degree each time a beam of light, for example, emitted by the position component 236 encounters a bore 234 in the rotor 104. In other embodiments, the positioning element formed within the rotor 104 may comprise a channel into which a tick fence (e.g., a thin sheet of metal or other material comprising one or more teeth or ticks) is inserted.

The rotor 104 may further comprise a drive channel 240 configured to receive a (substantially-annular) drive mechanism 242 (e.g., such a belt, rope, or other drive mechanism). Typically, the drive mechanism 242 is configured to rotate the rotor 104 and is operatively coupled to the drive channel 240. That is, the rotor 104 may be machined/manufactured with a channel 240 into which a drive mechanism 242, such as a belt, may be placed for rotating the rotor 104. By way of example, as described with respect to FIG. 1, a rotator (e.g., 116 in FIG. 1) may be positioned on a stationary side of the imaging modality and the drive mechanism 242 may be coupled to the rotator to cause the rotor 104 to rotate.

It may be appreciated that a belt is merely one example of a drive mechanism, and that other drive mechanisms are also contemplated. For example, the rotor 104 may be machined/manufactured with one or more teeth that serve as the drive mechanism 242 and are configured to contact or engage with teeth of a rotator. As such, the rotor 104 may not comprise a drive channel 240 because the drive mechanism 242 may be machined/manufactured to extend beyond a surface of the rotor 104, for example.

In one embodiment, the rotor 104 may be further machined or manufactured to comprise a bearing structure 244 (e.g., also referred to as a bearing surface or bearing face), that when coupled with a bearing structure 246 of the stator 110, for example, forms an assembled bearing, such as a ball bearing and/or an air bearing, configured to support at least some of the weight of the rotor 104.

To ground the rotor 104 and/or reduce static charge in the rotor 104, for example, the stator 110 may further comprise a static discharge component 248, such as one or more metal brushes configured to contact the rotor 104 and the rotor 104 may be configured to physically contact the one or more brushes. For example, in some embodiments, the rotor 104 may comprise a metal surface to which the one or more metal brushes are intended to make contact. Electrical charge created by the rotor 104 may be transmitted to the stator 110 (e.g., and grounded) via the static discharge component 248. It may be appreciated that although FIG. 2 and subsequent figures illustrate the brushes as coming into physical contact with the rotor 104, in another embodiment, the stator 110 may instead be in physical contact with the brushes. For example, the rotor 104 may comprise a static discharge arm to which brushes are connected and the brushes may come into contact with the stator 110.

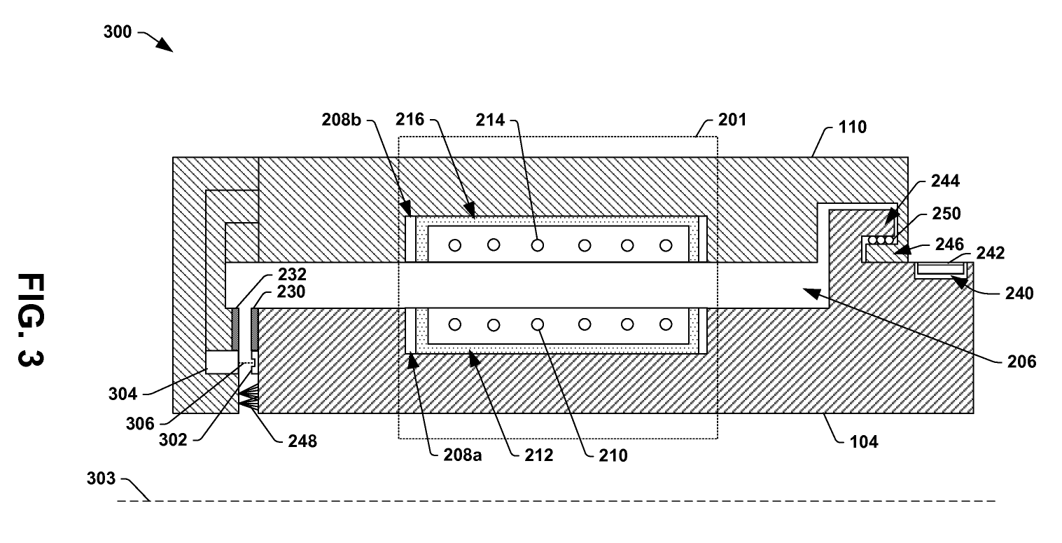

It may be appreciated that the arrangement of elements and/or components described with respect to FIG. 2 merely illustrates one example arrangement and is not intended to be interpreted as limiting the scope of the application, including the scope of the claims. That is, other arrangements for arranging the foregoing features of a rotor 104 and/or a stator 110 are also contemplated. For example, FIG. 3 illustrates a cross-sectional view of an example rotor 104 and a stator 110 separated by a substantially cylindrical airgap 206. In such an embodiment, the element 210 of the rotor 104 may be disposed on a radial surface of the rotor 104 and the element 214 of the stator 110 may be disposed on a radial surface of the stator 110.

While the arrangement of the elements and components are substantially similar to the components illustrated in FIG. 2 (except that the elements/components are disposed on opposite surfaces), it may be appreciated that the bearing structure 244 of the rotor 104 and/or the bearing structure 246 of the stator 110 may be slightly different given the difference in the orientation of the airgap 206. For example, in FIG. 3, the bearing structure 244 of the rotor 104 forms an (upside down) “L” shape that overlaps a backwards “L” shaped bearing structure 446 of the stator 110 (e.g., causing the bearing structures 244, 246 to lock together), and the ball bearings 250, for example, may be positioned between the “L” shaped bearing structure 244 and the “L” shaped bearing structure 246.

FIG. 3 illustrates a cross-sectional view 300 similar to the cross-sectional view 200 illustrated in FIG. 2 of the example rotor 104 and the stator 110 separated by a substantially planar airgap 206, wherein the rotor 104 is configured to rotate about an axis of rotation 303. In this embodiment, to facilitate ascertaining a rotation angle of the rotor 104 (e.g., relative to a zero-degree reference) during the rotation of the rotor 104, the rotor 104 may comprise one or more positioning elements 302 (e.g., slits, cavities, holes, notches, channels, etc.) and the stator 110 may comprise a position component 304 configured to determine the rotation angle of the rotor 104 based upon the one or more positioning elements (or vice-versa). In this example, the one or more positioning elements 302 may be located adjacent one of the antennas (e.g., the first antenna 230 in this embodiment) and/or may be formed within (e.g., integral to) an antenna structure in which the first antenna 230 is disposed.

The position component 304 may be configured to emit a light beam, infrared beam, or other electromagnetic radiation 306 in the direction of the positioning elements 302 to determine the rotation angle of the rotor 104. In this example, the position component 304 may be located adjacent one of the antennas (e.g., the second antenna 232 in this embodiment) and/or may be formed within (e.g., integral to) an antenna structure in which the second antenna 232 is disposed. Typically, the positioning elements 302 are spaced evenly around the rotor 104, and when the electromagnetic radiation 306 encounters a positioning element 302 (e.g., a notch, cavity, etc.) (e.g., or when electromagnetic radiation is not reflected back to the position component 304), it may be determined that the rotor 104 has rotated a predetermined distance. In an example, in one embodiment, the rotor 104 may comprise 360 positioning elements 302 spaced one-degree apart. As such, the position component 304 may determine that the rotor 104 has rotated one-degree each time a beam of light, for example, emitted by the position component 304 encounters a positioning element 302 in the rotor 104. In another embodiment, the number of positioning elements may be a function of a specified number of views to be acquired during a 360 degree rotation, for example.

In other embodiments, the positioning element formed within the rotor 104 may comprise a channel into which a tick fence (e.g., a thin sheet of metal or other material comprising one or more notches) is inserted. It is to be appreciated that the foregoing examples of how a positioning element may be constructed are provided merely as non-limiting examples and that other constructions of the positioning element are also contemplated.

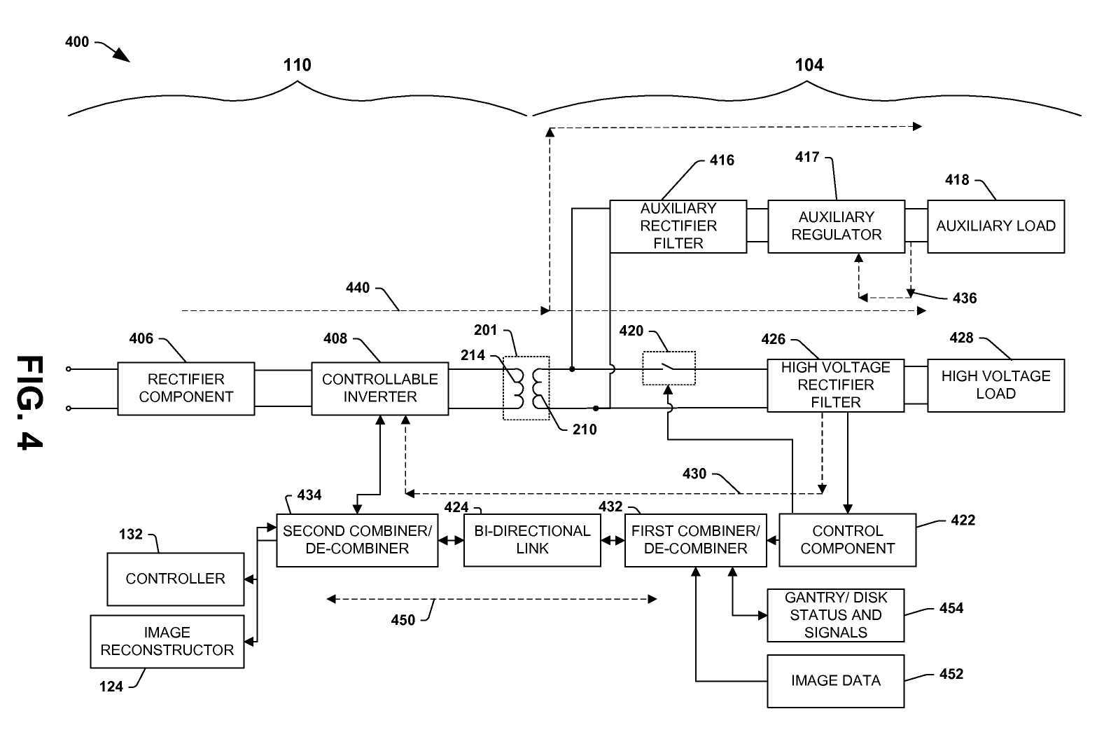

FIG. 4 illustrates a schematic block diagram of a system 400 configured to transfer power and information (e.g., data) between a stator 110 and a rotor 104 configured for relative movement. Power is provided to an auxiliary load 418 and a high voltage load 428 via a power transmission path 440. The auxiliary load 418 may comprise the detector array 106, control electronics, a spine heater, a tube heat exchanger, an anode driver, etc. The high voltage load 428 may comprise the radiation source 118, for example. As will be described in more detail below, an auxiliary regulator 417 is configured to self-regulate an output applied to the auxiliary load 418. Thus, an auxiliary control loop 436 is formed between the output of the auxiliary regulator 417 and the auxiliary load 418. Moreover, as will be described in more detail below, a high voltage rectifier filter 426 is configured to regulate its output based upon electrical characteristics, such as a frequency and/or amplitude, of an electrical signal received by the high voltage rectifier filter 426. In this regard, a high voltage control loop 430 is formed between the high voltage rectifier filter 426 and a controllable inverter 408 disposed on the stator 110. Control messages are provided by way of this high voltage feedback loop 430 to the controllable inverter 408, where the control messages inform/instruct the controllable inverter 408 how to modify a signal (e.g., referred to herein as a modulated signal) to achieve the desired output at the high voltage rectifier filter 426. Further, as will be described below, the control messages may be combined with other information, such as status messages and/or image data, to be transmitted between the rotor 104 and the stator 110 by way of a bi-directional link 424. The flow of information across the bi-directional link 424 is illustrated by arrow 450.

The system 400 comprises a rectifier component 406 configured to rectify an input electrical signal (e.g., yielded from an electric generator). That is, the rectifier component 406 may be configured to convert an alternating current (AC) input signal into a direct current (DC) signal. Moreover, the rectifier component 406 may be configured to alter other characteristics of the AC input signal to improve the quality of the DC signal relative to the AC input signal and/or to make adjustments to the AC input signal. For example, in an embodiment, the rectifier component 406 may adjust or correct a phase of the AC input signal.

The system 400 further comprises the controllable inverter 408 configured to receive the DC signal output by the rectifier component 406. The controllable inverter 408 (e.g., an inverter, high frequency inverter, high frequency resonant inverter, etc.) can invert the signal from the rectifier component 406 (e.g., converting the signal from DC to AC) to generate a modulated signal comprising an alternating current. The modulated signal is output from the controllable inverter 408 and transmitted to a primary winding 214 of the rotary transformer 201, where the modulated signal creates an electromagnetic field that induces an electrical signal on a secondary winding 210 of the rotary transformer 201. In an example, the primary winding 214 is disposed on the stator 110 while the secondary winding 210 is disposed on the rotor 104. The rotary transformer 201 delivers power between the stator 110 and the rotor 104, where the power is supplied to the auxiliary load 418 and the high voltage load 428.

One or more characteristics of the modulated signal, such as frequency and/or amplitude of the modulated signal, is set by the controllable inverter 408 based upon a desired voltage to be applied to the high voltage load 428. During periods when the high voltage load 428 is turned on, the one or more characteristics of the modulated signal may be adjusted based upon the voltage being applied to the high voltage load 428 and/or based upon ripples in the voltage applied to the high voltage load 428. For example, as will be described in more detail below, a control component 422 of the system 400 may receive a reference voltage from the high voltage rectifier filter 426 and may generate a control message indicative of characteristics of the second voltage applied to the high voltage load 428 and/or indicative of a desired change to one or more characteristics of the modulated signal (e.g., to alter a characteristic of the second voltage applied to the high voltage load 428). The control message may be delivered to the controllable inverter 408 through the control loop 430 formed between the high voltage rectifier filter 426 and the controllable inverter 408 (e.g., by way of a first combiner/de-combiner 432, a second combiner/decombiner 434, and the bi-directional link 424). The controllable inverter 408, in turn, sets/adjusts one or more characteristics of the modulated signal based upon the control message.

The rotary transformer 201 is configured to transfer power between the stator 110 and the rotor 104. Stated differently, the rotary transformer 201 is configured to transfer the modulated signal and/or characteristics thereof from the stator 110 to the rotor 104. It will be appreciated that for purposes of clarity, the signal may be referred to as the “modulated signal” when the signal is within the stator 110 or components attached thereto and may be referred to as a “first electrical signal” when the signal is within the rotor 104 or components attached thereto.

The rotary transformer 201 comprises the primary winding 214 and a secondary winding 210 disposed on the rotor 104). To supply power to the rotor 104, the rotary transformer 201 is configured to generate a first electrical signal on the secondary winding 210 based at least in part upon the modulated signal, which is passed through the primary winding 214 to generate an electrical field that induces a current in the secondary winding 210. Stated differently, the modulated signal is passed through the primary winding 214 to induce a current corresponding to the first electrical signal in the secondary winding 210. As such, the first electrical signal may be generated based on the frequency and the amplitude of the modulated signal. That is, the frequency of the first electrical signal may be a function of the frequency of the modulated signal. Similarly, the amplitude of the first electrical signal may be a function of the amplitude of the modulated signal.

In some embodiments, the secondary winding 210 is configured to increase (e.g., step up) or decrease (e.g., step down) a first voltage of the first electrical signal relative to a voltage of the modulated signal (e.g., and/or alter other characteristics of the first electrical signal relative to similar characteristics of the modulated signal). In other embodiments, the secondary winding 210 is configured to generate a first electrical signal that substantially matches the modulated signal. Thus, amplitude, frequency, and/or other characteristics of the first electrical signal output by the secondary winding 210 may substantially match amplitude, frequency, and/or other characteristics of the modulated signal, for example. In an example, the first electrical signal can be associated with a first voltage (e.g., 800 V).

It is to be appreciated that the instant application is not intended to be limited to a rotary transformer 201 for transferring power and that other power transfer apparatuses are contemplated. For example, in some embodiments, the rotary transformer 201 is replaced by slip ring, and the modulated signal may be transferred to the rotor 104 via a brush and ring assembly. Thus, the modulated electrical signal may be transferred to the rotor 104 via the slip ring. It will be appreciated that where the modulated signal is transferred to the rotor 104 via the slip ring, the modulated signal may be regarded as the first electrical signal upon arrival at the rotor 104.

Regardless of embodiment (e.g., rotary transformer, slip ring, etc.), the first electrical signal is channeled to at least two different circuits of (e.g., disposed on) the rotor 104. A first circuit is configured to deliver power from the rotary transformer 201 or other power transfer apparatus to the auxiliary load 418. A second circuit is configured to deliver power from the rotary transformer 201 or other power transfer apparatus to the high voltage load 428. In some embodiments, the first circuit comprises an auxiliary rectifier filter 416 and the auxiliary regulator 417 coupled between the rotary transformer 201 and the auxiliary load 418. The second circuit comprises a high voltage rectifier filter 426 coupled between the rotary transformer 201 and the high voltage load 428.

The auxiliary rectifier filter 416 is configured to receive the first electrical signal from the secondary winding 210 and to convert the first electrical signal from an AC signal to a DC signal. The auxiliary rectifier filter 416 may be a full-wave rectifier, half-wave rectifier, bridge rectifier etc.

The auxiliary regulator 417 is configured to receive the DC signal from the auxiliary rectifier filter 416 and adjust or regulate the first voltage that is applied to the auxiliary regulator 417. By way of example, a voltage associated with the DC signal may be substantially equal to the first voltage associated with the first electrical signal (e.g., 800 V), and the auxiliary regulator 417 may be configured to adjust (e.g., step down) the voltage to an operating voltage of the auxiliary load 418. For example, in some embodiments, the operating voltage is approximately 300-400 V. In the illustrated example, the auxiliary regulator 417 is illustrated as outputting a specified voltage to the auxiliary load 418. In other examples, one or more auxiliary regulators 417 may be provided that are electrically coupled to the auxiliary rectifier filter 416, with the one or more auxiliary regulators 417 configured to output a plurality of different specified voltages.

In some embodiments, the voltage output by the auxiliary regulator 417 is predefined based upon the operating voltage of the auxiliary load 418. Thus, the voltage that is output by the auxiliary regulator 417 is generated/adjusted independently of characteristics of the first electrical signal, such as independent of the frequency and/or the amplitude of the first electrical signal. A signal output by the auxiliary regulator 417 and applied to the auxiliary load 418 is, at times, referred to as a third electrical signal.

In some embodiments, the auxiliary regulator 417 is configured to self-regulate the voltage output by the auxiliary regulator 417. For example, the auxiliary regulator 417 uses the control loop 436 (e.g., a negative feedback control loop) to provide feedback to the auxiliary regulator 417 regarding an actual voltage output by the auxiliary regulator 417 so that the regulator can make adjustments to the output voltage. In some embodiment, the auxiliary regulator 417 comprises a buck or a boost converter.

The second circuit is configured to deliver power from the rotary transformer 201 or other power transfer apparatus to the high voltage load 428. In some embodiments, the second circuit comprises a switch 420, the high voltage rectifier filter 426, and the high voltage load 428.

The switch 420 is configured to selectively decouple the rotary transformer 201 or other power transfer apparatus from the high voltage load 428, thus inhibiting a flow of current between the rotary transformer 201 and the high voltage load 428 and/or between the rotary transformer 201 and the high voltage rectifier filter 426. For example, during periods when the high voltage load 428 is to be turned off or deactivated, the switch 420 may be opened via a signal from the control component 422, for example, to decouple the high voltage load 428 from the rotary transformer 201. In an example, the switch 420 can be disposed on the rotor 104.

By way of example, in a CT application, the switch 420 can be configured to control the emission of radiation from the high voltage load 428 (e.g., a radiation source). When the switch 420 is open (e.g., when the examination unit 108 in FIG. 1 is in prepare mode), power may not be supplied to the high voltage load 428 and radiation may not be emitted. When the switch 420 is closed (e.g., when the examination unit 108 in FIG. 1 is in shoot mode), power may be supplied from the secondary winding 210 to the high voltage load 428, via the high voltage rectifier filter 426, to activate the radiation source and facilitate the generation/emission of radiation. In some embodiments, as will be further described below, the opening or the closing of the switch 420 by the control component 422 is controlled by the controller 132, which transmits control messages to the control component 422 by way of the bi-directional link 424. For example, the switch 420 may comprise one or more transistors and the control component 422 may apply a voltage to a gate(s) of the one or more transistors to enable the transistors to conduct (e.g., thus closing the switch and allowing the first electrical signal to flow through the switch and be received at the high voltage rectifier filter 426).

The high voltage rectifier filter 426 is coupled to the secondary winding 210 and is configured to receive the first electrical signal, associated with the first voltage, from the secondary winding 210 (e.g., when the switch 420 is closed). The high voltage rectifier filter 426 is configured to, among other things, rectify the first electrical signal. In this way, the first electrical signal can be converted from an AC signal to a DC signal that may be desired and/or used by the high voltage load 428. A signal output by the high voltage rectifier filter 426 and applied to the high voltage load 428 is, at times, referred to as a second electrical signal.

The high voltage rectifier filter 426 is also configured to generate (e.g., set and/or adjust) a voltage output by the high voltage rectifier filter 426 and supplied to the high voltage load 428. In some embodiments, unlike the auxiliary regulator 417, which self-regulates the voltage applied to the auxiliary load 418, the high voltage rectifier filter 426 is not self-regulating. For example, in some embodiments, the high voltage rectifier filter 426 uses the frequency, amplitude, and/or other characteristics of the first electrical signal input into the high voltage rectifier filter 426 to generate the voltage applied to the high voltage load 428. For example, the high voltage rectifier filter 426 may use the frequency and/or amplitude of the first electrical signal to determine a degree to which a first voltage associated with the first electrical signal, input into the high voltage rectifier filter 426, is to be stepped up to generate an output voltage applied to the high voltage load 428 (e.g., stepping up the voltage from 800 V to 60 kV or more). In some embodiments, the amplitude and/or frequency of the first electrical signal may be varied to change a property of the second electrical signal and/or to reduce a voltage ripple associated with the second electrical signal. In an embodiment, the voltage applied to the high voltage load is greater than a voltage applied to the high voltage rectifier filter 426 by the secondary winding 210. As such, the high voltage rectifier filter 426 can step up the first voltage based on at least one of the frequency or the amplitude of the first electrical signal to generate the second electrical signal.

To facilitate adjustments of the output of the high voltage rectifier filter, the control loop 430 is formed between the high voltage rectifier filter 426 and the controllable inverter 408. By way of example, the second electrical signal output by the high voltage rectifier filter 426 and/or a reference signal generated therefrom may be applied to the control component 422. Based upon the voltage applied to the control component 422, the control component 422 may generate a control message describing one or more properties of the second electrical signal (e.g., describing a numerical value of the second voltage associated with the second electrical signal and/or reference voltage of the reference signal) and/or describing how a characteristic of the modulated signal should be modified in light of the voltage applied to the control component 422. Such a control message may be communicated to the controllable inverter 408 via the bi-directional link 424.

In some embodiment, the control message is further combined or packaged with other data, such as image data 452, status data 454, etc. prior to transmission across the bi-directional link 424 by the first combiner/de-combiner 432. In such embodiments, the second combiner/de-combiner 434 may be disposed on the stator 110 to de-combine (e.g., separate) the control message from the image data 452, status data 454, etc. For example, the control message(s) generated by the control component 422 may be combined with image data and/or status message to be transmitted across the bi-directional link 424. The first combiner/de-combiner 432 may receive the control message(s), image data, etc. and combine/package the data for transmission across the bi-direction link 424. The second combiner/de-combiner 432 may de-combine/deconstruct the data and provide the control message(s) to the controllable inverter, the image data to the image reconstructor, etc.

In some embodiments, if the second voltage associated with the second electrical signal has an undesired characteristic (e.g., the voltage ripple is too large, the root mean square (rms) voltage is too low or too high, etc.), the controllable inverter 408 is configured to determine how one or more characteristics of the modulated signal (e.g., frequency, amplitude, etc.) should be modified to improve the undesired characteristic based upon the control message. If the second voltage associated with the second electrical signal is within an acceptable tolerance (e.g., has no undesired characteristics), the controllable inverter 408 continues to output the modulated signal according to the status quo (e.g., maintains the frequency, amplitude, etc. of the modulated signal). In other embodiments, where the control message specifies desired characteristics of the modulated signal, the controllable inverter 408 merely receives the control message by way of the control loop 430 from the control component 422 and adjusts the modulated signal to have the desired characteristics. Because the output of the high voltage rectifier filter 426 is a function of this modulated signal, or rather a signal derived therefrom, the output of the high voltage rectifier filter is adjusted based upon the adjustment to the modulated signal output from the controllable inverter 408, for example.

It will be appreciated that example system 400 of FIG. 4 merely illustrates example components, and is not intended to be viewed in a limiting manner as necessarily specifying and/or illustrating all of the components of the system. Moreover, at least some of the components described herein may be optional.

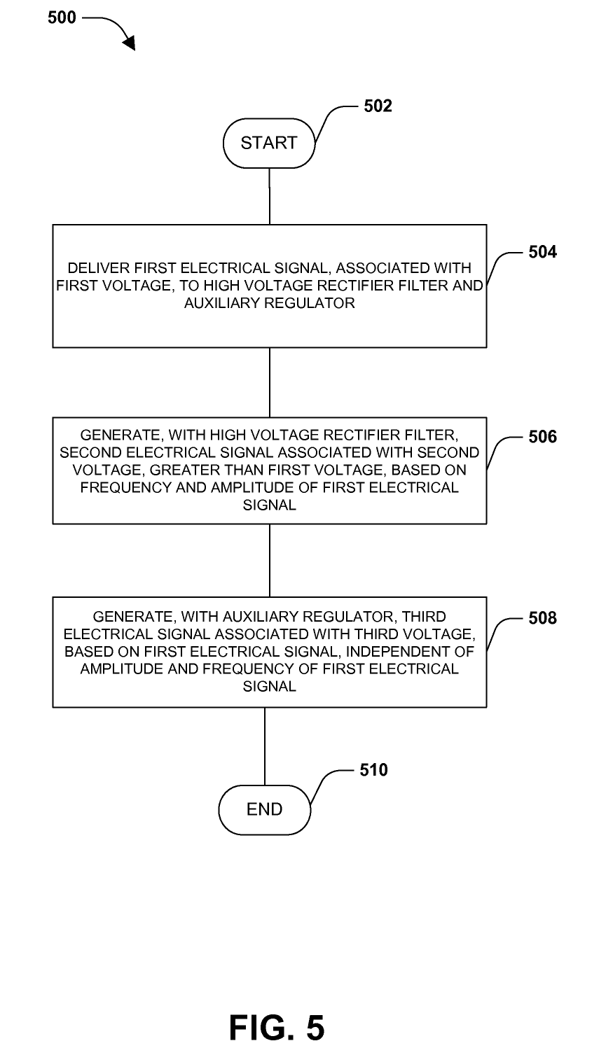

Referring to FIG. 5, a flow diagram of an example method 500 for concurrently supplying power to a high voltage load and to an auxiliary load is provided.

The example method 500 begins at 502 when a first electrical signal is generated. In an example, to supply power to the rotor 104, the rotary transformer 201 can generate the first electrical signal on the secondary winding 210. The first electrical signal is based, at least in part, upon the modulated signal that is passed through the primary winding 214.

At 504, the first electrical signal, associated with a first voltage, is delivered to the high voltage rectifier filter 426 and an auxiliary rectifier filter 416. In an example, the first electrical signal is delivered to at least two different circuits of the rotor 104: a first circuit, which delivers power to the auxiliary load 418, and a second circuit, which delivers power to the high voltage load 428.

At 506, the high voltage rectifier filter 426 generates a second electrical signal associated with a second voltage, which is greater than the first voltage, based on a frequency and an amplitude of the first electrical signal. In an example, the high voltage rectifier filter 426 is not self-regulating. For example, the high voltage rectifier filter 426 can use the frequency and the amplitude of the first electrical signal input into the high voltage rectifier filter 426 to generate the second voltage associated with the second electrical signal.

At 508, an auxiliary regulator coupled to the auxiliary rectifier filter 416 generates a third electrical signal associated with a third voltage, which is based on the first electrical signal, independent of the amplitude and the frequency of the first electrical signal. In an example, the third voltage output by the auxiliary regulator 417 is generated/adjusted independently of characteristics of the first electrical signal, such as the frequency and/or the amplitude of the first electrical signal.

The example method 500 ends at 510.

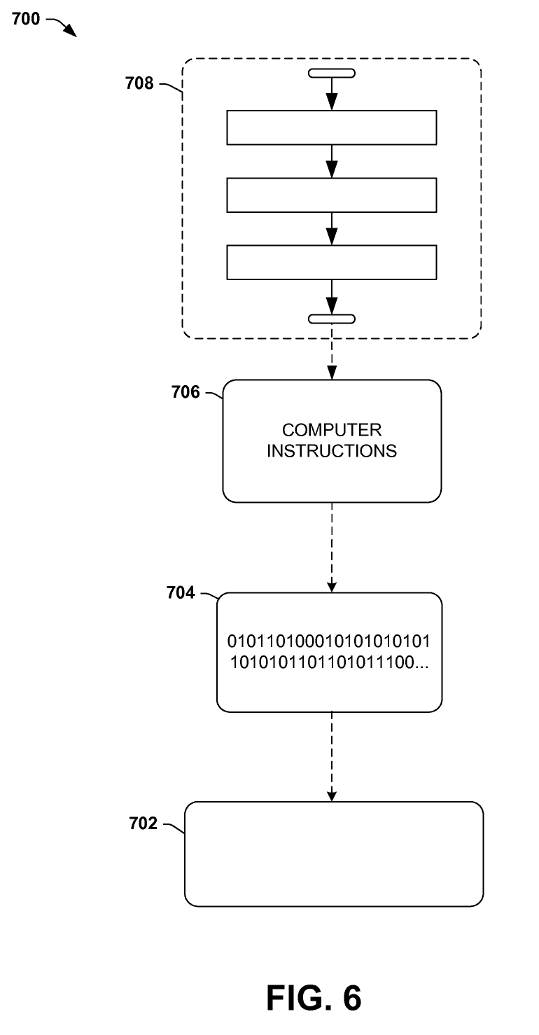

Still another embodiment involves a computer-readable medium comprising processor-executable instructions configured to implement one or more of the techniques presented herein. An example computer-readable medium that may be devised in these ways is illustrated in FIG. 6, wherein the implementation 600 comprises a computer-readable medium 602 (e.g., a flash drive, CD-R, DVD-R, application-specific integrated circuit (ASIC), field-programmable gate array (FPGA), a platter of a hard disk drive, etc.), on which is encoded computer-readable data 604. This computer-readable data 604 in turn comprises a set of processor-executable instructions 606 configured to operate according to one or more of the principles set forth herein. In one such an embodiment 600, the processor-executable instructions 606 may be configured to perform a method 608 when executed via a processing unit, such as at least some of the example method 500 of FIG. 5. In another such embodiment, the processor-executable instructions 606 may be configured to implement a system, such as at least some of the example environment 100 of FIG. 1. Many such computer-readable media may be devised by those of ordinary skill in the art that are configured to operate in accordance with one or more of the techniques presented herein. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

Although the subject matter has been described in language specific to structural features or methodological acts, it is to be understood that the subject matter of the appended claims is not necessarily limited to the specific features or acts described above. Rather, the specific features and acts described above are disclosed as embodiment forms of implementing at least some of the claims.

Various operations of embodiments are provided herein. The order in which some or all of the operations are described should not be construed to imply that these operations are necessarily order dependent. Alternative ordering will be appreciated given the benefit of this description. Further, it will be understood that not all operations are necessarily present in each embodiment provided herein. Also, it will be understood that not all operations are necessary in some embodiments.

Moreover, “exemplary” is used herein to mean serving as an example, instance, illustration, etc., and not necessarily as advantageous. As used in this application, “or” is intended to mean an inclusive “or” rather than an exclusive “or”. In addition, “a” and “an” as used in this application are generally be construed to mean “one or more” unless specified otherwise or clear from context to be directed to a singular form. Also, at least one of A and B and/or the like generally means A or B or both A and B. Furthermore, to the extent that “includes”, “having”, “has”, “with”, or variants thereof are used, such terms are intended to be inclusive in a manner similar to the term “comprising”. The claimed subject matter may be implemented as a method, apparatus, or article of manufacture (e.g., as software, firmware, hardware, or any combination thereof).

As used in this application, the terms “component,” “module,” “system”, “interface”, and the like are generally intended to refer to a computer-related entity, either hardware, a combination of hardware and software, software, or software in execution. For example, a component may be, but is not limited to being, a process running on a processor, a processor, an object, an executable, a thread of execution, a program, and/or a computer. By way of illustration, both an application running on a controller and the controller can be a component. One or more components may reside within a process and/or thread of execution and a component may be localized on one computer and/or distributed between two or more computers.

Furthermore, the claimed subject matter may be implemented as a method, apparatus, or article of manufacture using standard programming and/or engineering techniques to produce software, firmware, hardware, or any combination thereof to control a computer to implement the disclosed subject matter. The term “article of manufacture” as used herein is intended to encompass a computer program accessible from any computer-readable device, carrier, or media. Of course, those skilled in the art will recognize many modifications may be made to this configuration without departing from the scope or spirit of the claimed subject matter.

Further, unless specified otherwise, “first,” “second,” and/or the like are not intended to imply a temporal aspect, a spatial aspect, an ordering, etc. Rather, such terms are merely used as identifiers, names, etc. for features, elements, items, etc. For example, a first channel and a second channel generally corresponds to channel A and channel B or two different or two identical channels or the same channel.

Although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure. In addition, while a particular feature of the disclosure may have been disclosed with respect to only one of several implementations, such feature may be combined with one or more other features of the other implementations as may be desired and advantageous for any given or particular application.

1. A computed tomography (CT) imaging modality comprising:
a stator;
a rotor configured to rotate relative to the stator;
a rotary transformer comprising a primary winding disposed on the stator and a secondary winding disposed on the rotor, the rotary transformer configured to deliver power between the stator and the rotor;
a high voltage rectifier filter coupled to the secondary winding and configured to receive a first electrical signal from the secondary winding, wherein the first electrical signal is associated with a first voltage and the high voltage rectifier filter is configured to generate a second electrical signal associated with a second voltage, greater than the first voltage, based on a frequency and an amplitude of the first electrical signal; and
an auxiliary regulator coupled to the secondary winding and configured to generate a third electrical signal based on the first electrical signal, the third electrical signal associated with a third voltage, the auxiliary regulator configured to self-regulate the third voltage.

2. The CT imaging modality of claim 1, wherein the auxiliary regulator is configured to generate the third electrical signal independent of the amplitude of the first electrical signal.

3. The CT imaging modality of claim 1, wherein the auxiliary regulator is configured to generate the third electrical signal independent of the frequency of the first electrical signal.

4. The CT imaging modality of claim 1, wherein the high voltage rectifier filter is configured to step up the first voltage based on at least one of the frequency or the amplitude of the first electrical signal to generate the second electrical signal.

5. The CT imaging modality of claim 1, wherein the high voltage rectifier filter is configured to step up the first voltage based on the frequency and the amplitude of the first electrical signal to generate the second electrical signal.

6. The CT imaging modality of claim 1, wherein the auxiliary regulator comprises a buck or a boost converter.

7. The CT imaging modality of claim 1, comprising a control component coupled to the high voltage rectifier filter and configured to generate a control message for conveyance from the rotor to the stator, the control message describing one or more properties of the second electrical signal.

8. The CT imaging modality of claim 7, comprising a controllable inverter coupled to the primary winding and configured to adjust at least one of an amplitude or a frequency of a modulated signal based on the control message, the modulated signal transmitted to the primary winding.

9. The CT imaging modality of claim 8, wherein the amplitude of the first electrical signal is a function of the amplitude of the modulated signal and the frequency of the first electrical signal is a function of the frequency of the modulated signal.

10. The CT imaging modality of claim 1, comprising a switch disposed on the rotor and configured to selectively decouple the high voltage rectifier filter from the secondary winding.

11. The CT imaging modality of claim 1, comprising a controllable inverter disposed on the stator and coupled to the primary winding, the controllable inverter configured to adjust a frequency and an amplitude of a modulated signal transmitted to the primary winding.

12. The CT imaging modality of claim 11, wherein the first electrical signal is generated based on the frequency and the amplitude of the modulated signal.

13. A computed tomography (CT) imaging modality comprising:
a stator;
a rotor configured to rotate relative to the stator;
a rotary transformer comprising a primary winding disposed on the stator and a secondary winding disposed on the rotor, the rotary transformer configured to deliver power between the stator and the rotor;
a high voltage rectifier filter coupled to the secondary winding, the high voltage rectifier filter configured to:
receive a first electrical signal, associated with a first voltage, from the secondary winding; and
step up the first voltage based on a frequency and an amplitude of the first electrical signal to generate a second electrical signal associated with a second voltage, the second voltage greater than the first voltage; and
an auxiliary regulator coupled to the secondary winding and configured to generate a third electrical signal based on the first electrical signal, the third electrical signal associated with a third voltage.

14. The CT imaging modality of claim 13, wherein the auxiliary regulator is configured to self-regulate the third voltage.

15. The CT imaging modality of claim 14, wherein the auxiliary regulator is configured to generate the third electrical signal independent of the amplitude of the first electrical signal.

16. The CT imaging modality of claim 14, wherein the auxiliary regulator is configured to generate the third electrical signal independent of the frequency of the first electrical signal.

17. A system, comprising:
a high voltage rectifier filter coupled to a secondary winding of a rotary transformer, the high voltage rectifier filter configured to:
receive a first electrical signal, associated with a first voltage, from the secondary winding; and
step up the first voltage based on at least one of a frequency or an amplitude of the first electrical signal to generate a second electrical signal associated with a second voltage; and
an auxiliary regulator coupled to the secondary winding and configured to generate a third electrical signal based on the first electrical signal, the third electrical signal associated with a third voltage, the third voltage less than the first voltage.

18. The system of claim 17, wherein the high voltage rectifier filter is configured to step up the first voltage based on the frequency and the amplitude of the first electrical signal.

19. The system of claim 17, wherein the auxiliary regulator comprises a buck converter.

20. The system of claim 17, comprising:
a control component coupled to the high voltage rectifier filter and configured to generate a control message for conveyance from a rotor to a stator, the control message describing one or more properties of the second electrical signal; and
a controllable inverter coupled to a primary winding of the rotary transformer and configured to adjust at least one of an amplitude or a frequency of a modulated signal based upon the control message, the modulated signal transmitted to the primary winding and the first electrical signal generated based on the frequency and the amplitude of the modulated signal.

21-26. (cancelled)
